(19) United States
(12) Patent Application Publication
(10) Pub. No.: US 2024/0400961 A1
(43) Pub. Date: Dec. 5, 2024

(54) CELL SHEET BIOREACTOR

(71) Applicant: CAROMEATS INC., Kitchener (CA)

(72) Inventor: Alireza Shahin-Shamsabadi, Kitchener (CA)

(21) Appl. No.: 18/611,274

(22) Filed: Mar. 20, 2024

Related U.S. Application Data
(60) Provisional application No. 63/504,827, filed on May 30, 2023.

(51) Int. Cl. C12M 1/12; C12M 1/02; C12M 3/00

(52) U.S. Cl. CPC C12M 25/02; C12M 23/04; C12M 23/44; C12M 27/00

(57) ABSTRACT

A bioreactor contains one or more substrates or membranes. The membranes may be provided as part of trays having walls to contain seed cells on the membranes. Multiple trays may be formed in a stack and immersed in liquid media in the bioreactor. Gaps are provided between the trays to allow liquid media to flow horizontally into or out of the stack. Optionally, the bioreactor has an impeller configured to direct liquid media laterally. An optional cell seeding system allows a suspension of cells to be placed on a substrate while the substrate is in the bioreactor, for example via the gaps. Optionally, the bioreactor has a filtration or sorption system to remove contaminants from, or optionally add molecules, to the liquid media. The bioreactor may contain circulation and oxygenation systems to distribute gasses and nutrients as well as to remove inhibitory or toxic by-products of cellular metabolism.

### CROSS-REFERENCE TO RELATED APPLICATIONS

This application claims priority to U.S. Provisional Application No. 63/504,827,filed May 30, 2023, which is incorporated herein by reference.

### FIELD

This specification relates to cell culture bioreactors, and to methods of growing cells, for example cell sheets or derivatives of cell sheets.

### BACKGROUND

International Publication Number WO 2022/000086 A1, Self-Assembled Cell Sheet Constructs and Methods of Making Thereof, describes a method of making a cell sheet construct. The method includes plating a plurality of cells on a substantially flat surface. The plurality of cells are grown to an at least 80% confluent cell sheet with intercellular linkages. A series of cell culture media having different pH are applied to obtain a substantially planar untethered cell sheet.

### INTRODUCTION

This specification describes a cell culture bioreactor. The bioreactor may be used, for example, to grow cell sheets or derivatives of cell sheets.

The bioreactor contains one or more substrates, alternatively called membranes. In some examples, a membrane has a wall to contain seed cells on the membrane. In some examples, the membrane provides a tray, or is combined with a frame to provide a tray. The tray may support the membrane and/or inhibit membrane warping. One or more trays can be immersed in a liquid medium in the bioreactor. Multiple trays may be formed into a stack. Gaps are provided between trays to allow liquid media to flow horizontally into or out of the stack. An optional cell seeding system allows a suspension of cells to be placed on a membrane while the membrane is in the bioreactor, for example via the gaps.

In some examples, the bioreactor has an impeller configured to direct liquid media laterally into the gaps. For example, the impeller may be located on a sidewall of the bioreactor or rotate around a horizontal axis.

In some examples, the bioreactor has a sorption or filtration system to remove contaminants. The sorption system may be in direct communication with a medium in the bioreactor. Optionally the sorption unit may also add molecules to the liquid media.

### BRIEF DESCRIPTION OF FIGURES

FIG. 1A shows an isometric view of a tray, including a membrane and a frame, for a bioreactor.

FIG. 1B shows an isometric view of a stack of the trays of FIG. 1A.

FIG. 1C shows a side view of the stack of FIG. 1B.

FIG. 2 shows a vertical (side view) cross section of a first bioreactor including the stack of FIGS. 1B and 1C.

FIGS. 3A, 3B and 3C show vertical (side view) cross sections of a second bioreactor also having a liquid handling module for cell addition in three states: cell growth, cell seeding and filling.

FIGS. 4A and 4B show vertical (side view) and horizontal (top view) cross sections of a third bioreactor also having a filtration unit and a media homogenizer.

### DETAILED DESCRIPTION

Cell sheets may be grown on a substantially two-dimensional substrate or membrane. The word “substantially” is used to indicate that the substrate may have surface topography, for example a pattern of grooves with a spacing of 1000 micrometers or less between adjacent grooves, while still being substantially two-dimensional. In some examples, the process of sheet formation starts with seeding cells, such as progenitor or stem cells, on the membrane to about 60-90% confluence. The seed cells may be supplied to the membrane by way of a suspension of cells in a growth medium or differentiation medium. The cells settle onto the membrane and adhere to it. The cells are grown to form a substantially confluent layer over the substrate. Optionally, a differentiation media may be added to the vessel to differentiate the cells. After forming a first layer, more cells may be added to the vessel generally as described for the first layer. The new cells adhere on top of the first layer of cells and attach to them or fuse with them, optionally while differentiating. The step of adding new cells may be repeated until a multilayer cell construct is created. The multilayer cell construct may be removed from the membrane in the form of a sheet, or remodeled into another form of cell construct.

Optionally, one or more media added to grow or differentiate the cells, or a separate medium added to the vessel, may contain components that increases extracellular matrix (ECM) production by the cells. For example, an ascorbate or a crowding agent, such as polyethylene glycol or carrageenan, may be added. Additional or alternatively, a crosslinking agent such as lignin or tannic acid may be added to strengthen the ECM.

Once cells have grown, and optionally differentiated, in a sufficient number of layers (e.g. 3-10) and have produced enough ECM, they can be detached from the membrane as a sheet that includes both the cells and the ECM produced by the cells. The ECM is a network of macromolecules that are entangled with the cells. In some examples, the process of sheet formation involves scraping the edges of the membrane to lift off some of the cells. The whole sheet can be released by grabbing an edge and slowly pealing it off. Alternatively, shaking the membrane or continued incubation of the cell may release a cell sheet. In other examples, a cell sheet is removed by activating a responsive surface of the substrate. In other examples, a cell sheet is removed by adding a chemical agent such as an enzyme to degrade a bond between the cell sheet and the substrate. In other examples, a series of cell culture media having different pH are applied to release a cell sheet.

In some examples, the cell sheet can be remodelled into a different shape. A remodelled derivative of a cell sheet may be formed in the original culture vessel or in a different vessel. In some examples, multiple cell sheets are stacked together to form thicker cell sheets. In some examples, a cell sheet is attached to anchors and cultivated such that the cells remodel into a construct attached to the anchors.

In other methods, cells are grown in a three-dimensional substrate, for example a gel. Parts of the gel provide a synthetic component of the ECM as the cells multiply.

Various aspects of membranes, membranes 14 as described further below, cell culture and cell sheet formation and cell sheet remodeling are described in U.S. patent application Ser. No. 17/882,693 (now U.S. Pat. No. 11,718,830) Silicone-Based Membrane Surface Chemistry and Topography Control for Making Self-Assembled Cell Sheets; International application no: PCT/CA2023/050,779 (Publication No: WO 2023/240,336) Device and Method for Making Cell Sheets; and, U.S. Application No. 63/591,004 Anchored Cell Sheet Construct, which are incorporated by reference.

A bioreactor as described herein contains one or more trays for cell culture. A tray provides a vessel for cell culture. In some examples, a tray is generally dish-shaped, having a bottom surrounded by a wall. A wall may be, for example, a circular wall or a multi-segmented polygonal wall. The height of the wall may be in the range of 3-20 mm high, which is enough to hold a volume of a medium sufficient for seeding a layer of cells without being overly large. The wall is adapted to retain a medium in the tray when the tray is held in a substantially horizontal orientation but may optionally allow a medium to be poured from the tray. The bottom of the tray may provide a substrate for cell growth. Alternatively, a separate substrate may be inserted into the tray.

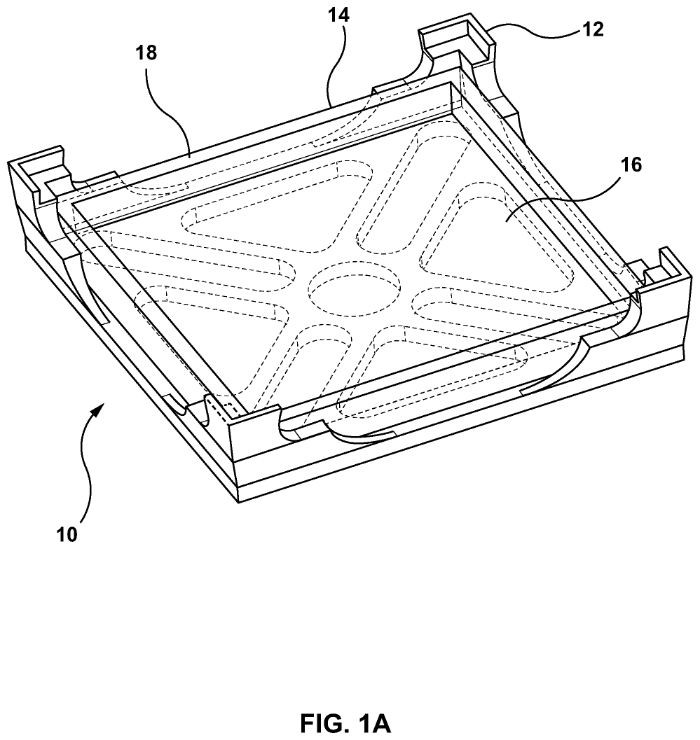

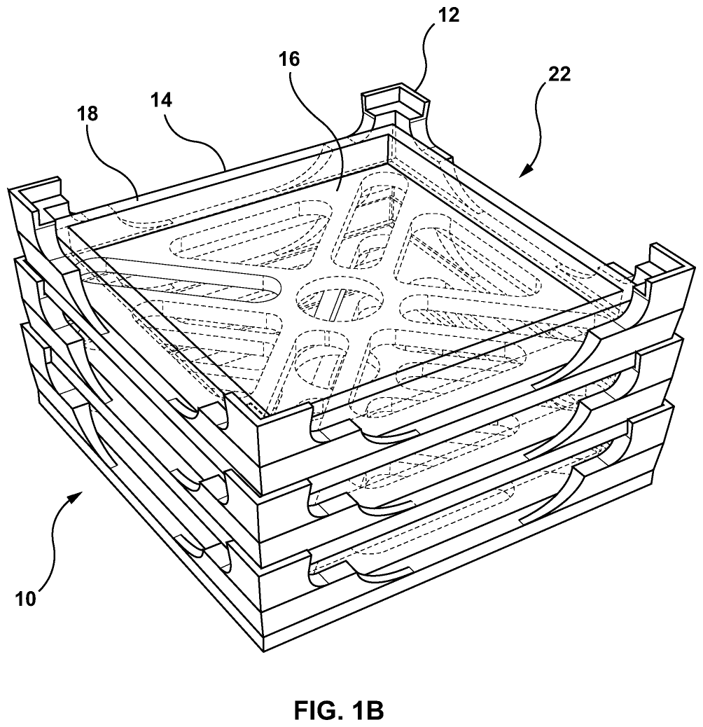

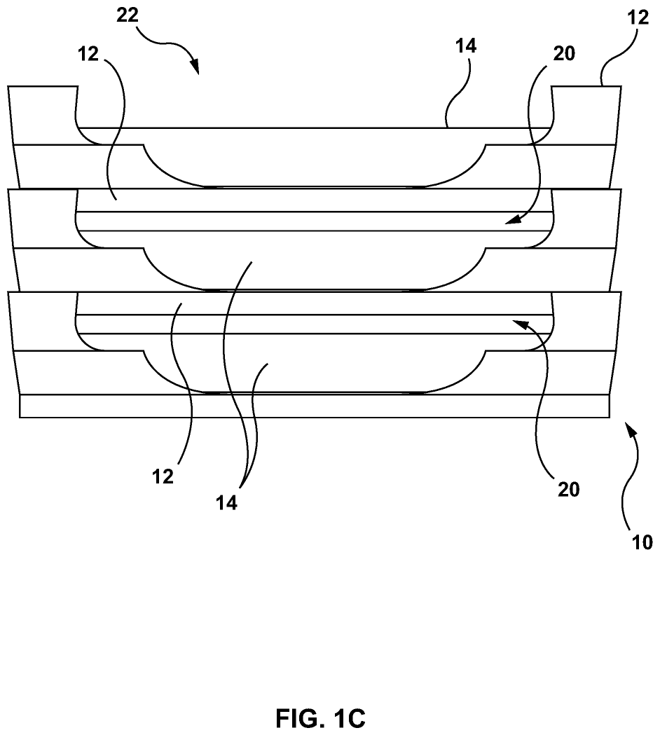

Referring to FIGS. 1A, 1B and 1C, in some examples a vessel for cell culture is provided by a tray 10. The tray 10 in this example includes a frame 12 and a membrane 14. The bottom of the frame 12 has outer dimensions that are less than the inner dimensions of the top of the frame 12. In this ways, multiple frames 12 may be stacked together with an upper frame 12 partially inserted into a lower frame 12 as shown in FIGS. 1B and 1C. Alternatively, a stack of frames 12 may be held together by way of a separate frame or holder, with or without one frame 12 inserted into another frame 12. For example, a holder may be in the form of a rack having a set of supports allowing trays 12 to slide into or out of the rack individually.

The frame 12 holds and supports a membrane 14. The membrane 14 is suitable for cell attachment. The membrane 14 is capable of holding a liquid and pores are not intentionally formed in it, although the membrane may be inherently gas permeable. The membrane 14 provides a substrate for cell culture. In some examples, the membrane 14 is made from a resin, for example a thermoplastic or thermosetting resin, that is cast or molded as a liquid and then cured or hardened to form a solid. In other examples, the membrane 14 is made, for example by cutting, bending or machining, from an already solid material.

The membrane 14 has a bottom 16 and a wall 18. The bottom 16 provides the substrate for growing cells. The walls 18 retain a suspension of cells in a liquid medium over the bottom 16 during cell seeding. In the example shown, the bottom 16 and the wall 18 are contiguous, although they are not necessarily the same material. For example, the bottom 16 may be cast from a silicone resin such as PDMS while the wall 18 is cast from PBAT. In other examples, the bottom 16 and the wall 18 may be two or more separate elements fastened together. In other examples, the wall 18 is a component of the frame 12 rather than the membrane 14. In other examples, the frame 12 and membrane 14 are a single piece.

In some examples, the bottom 16 is made of silicone, for example PDMS. The silicone may be treated, for example with tannic acid or lignin, to make it less hydrophobic and more suitable for cell culture.

In some examples, the bottom 16 is textured, or provided with topographical patterns, on its upper surface. At least an initial layer of cells may be aligned according to the pattern of the bottom 16. The pattern may include grooves with a spacing of 0.01-500 microns between adjacent grooves. Optionally, grooves of multiple sizes may be provided, including some grooves with a spacing of 5-50 microns between adjacent grooves. Despite the texturing or topographical patterns, the bottom 16 remains substantially flat in the sense that a liquid no more than 1-3 mm deep at any point may cover then entire bottom 16 when the bottom 16 is oriented horizontally.

The tray 10 may be of any shape, for example round, square or rectangular. The tray 10 optionally has a width or diameter in the range of 10 mm to 100 mm or more.

FIGS. 1B and 1C show multiple trays 10 assembled into a stack 22. As best seen in FIG. 1C, a gap 20 is provided between the tops of the wall 18 of a lower tray 10 and the bottom of an upper tray 10 of each pair of trays 10. The gaps 20 provide an opening for materials, for example media or cells, to be flow into or out of the stack 22, preferably into and out of each tray 10.

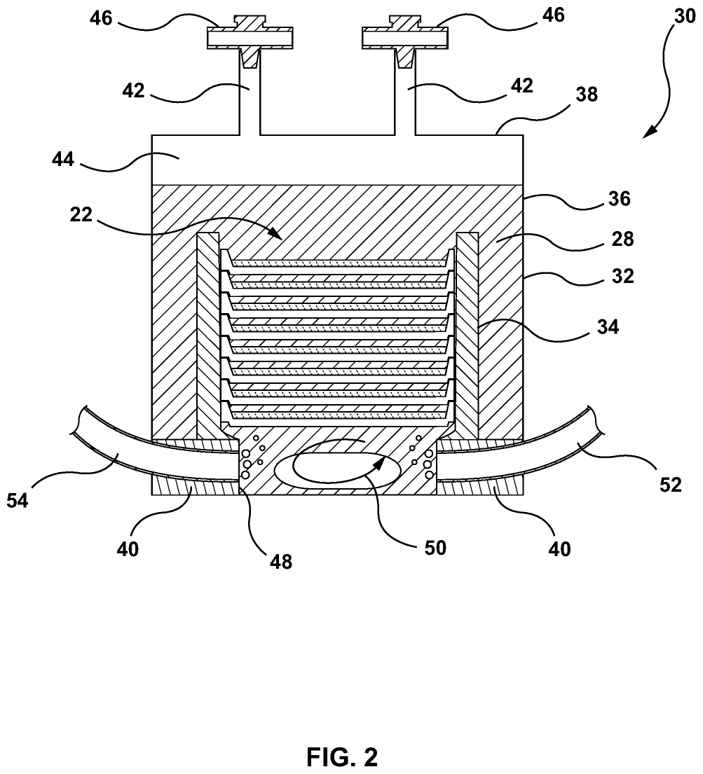

FIG. 2 shows a first bioreactor 30. The first bioreactor 30 has a shell 32, which may include a top 38, base 40 and one or more walls 36. The shell 32 defines a plenum for holding a liquid medium 28 and one or more trays 10. The first bioreactor 30 may contain a stack 22 of multiple trays 10. The one or more trays 10 are immersed in the medium 28 in bioreactor 30.

In the example shown, four posts 34 extend upwards from the base 40 of the first bioreactor 30. The posts 34 engage with the corners of one or more trays 10, or a stack 22. The posts 34 locate the trays 10 laterally in the first bioreactor 30. Optionally, the posts 34 may also support the trays 10 such that a bottom of a tray 10 or stack 22 is displaced from the top of the base 40 of the bioreactor 30.

The shell 32, for example the top 38, may have one or more ports 42. Ports 42 can be used, for example, to allow a gas to flow out from a headspace 44 of the bioreactor 30 to the atmosphere. Gas outlet ports 42 preferably have filters 46 to avoid contamination of the first bioreactor 30. The same or other ports 42 can be used, for example, for filling, emptying, sampling, or sensors. Optionally, sensors may be provided to monitor concentrations of one or more elements in the media, for example oxygen, glucose, lactic acid and ammonia. Sensors may also be provided to measure, for example, pH or temperature. Ports 42 may also be used to add agents, for example for pH adjustment, or to replenish liquid media or add nutrients or growth factors.

The base 40 has a recess 48 with an impeller 50. The impeller 50 may be rotated, for example, by way of a magnet or ferrous disk on the impeller 50 coupled with a magnet or ferrous disk on a motor below the base 40. The base 40 also has a first gas inlet 52 and optionally a second gas inlet 54. The gas inlets 52, 54 allow one or more gasses to be added to the first bioreactor 30, optionally into the recess 48 or otherwise near the impeller 50.

To support respiration of cells growing on a tray 10, one or more separate gasses or a blend of gasses flows into the base 40. The gasses typically include oxygen and carbon dioxide. Carbon dioxide may optionally be used to control the pH within the first bioreactor 30 where a bicarbonate buffer is used. The gasses are pumped into the first bioreactor 30 while the impeller 50 rotates at a desired speed to mix the gasses with liquid media 28 in the first bioreactor 30 and to circulate liquid media 28 around the trays 10. As mentioned above, the gaps 20 between trays 10 in a stack 22 allow the liquid media 28 to flow laterally into or out of the stack 22.

In this example, the top 38 of the first bioreactor 30 is removable. For seeding purposes the stack 22 is removed from the first bioreactor 30 and the trays 10 are de-stacked in a sterile environment. Cells are seeded on each tray 10 individually outside of the first bioreactor 30. Once cells show adhesion to the membranes 14 (30 minutes to a few hours depending on cell type), the trays 10 are re-stacked and the stack 22 is moved back into the first bioreactor 30.

The first bioreactor 30 may be heated, for example to the normal body temperature of an animal that cells are derived from. In some examples, the first bioreactor 30 is placed on a heater or wrapped with a heat jacket covering the walls 36 of the first bioreactor 30. In other examples, the entire first bioreactor 30 is located in an incubator or a heated biosafety cabinet.

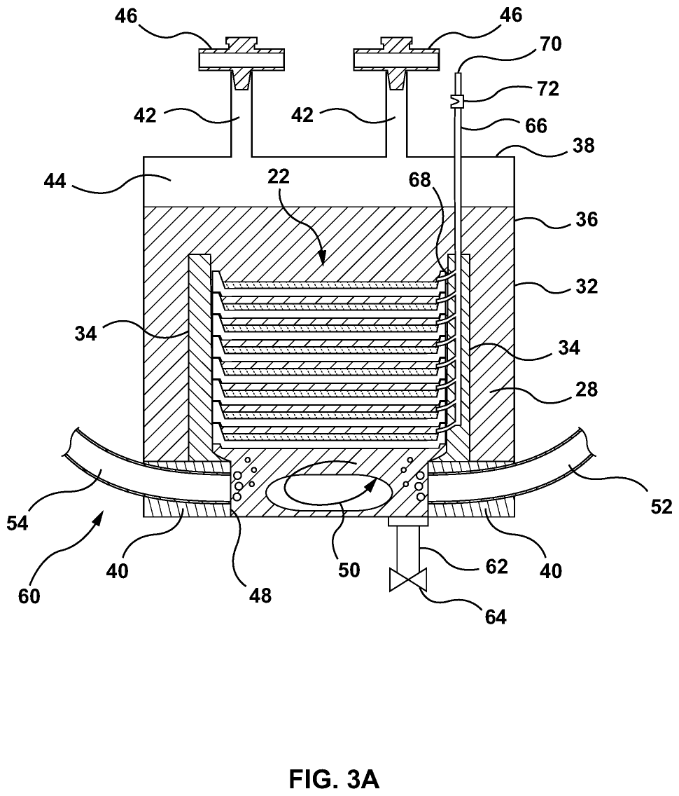

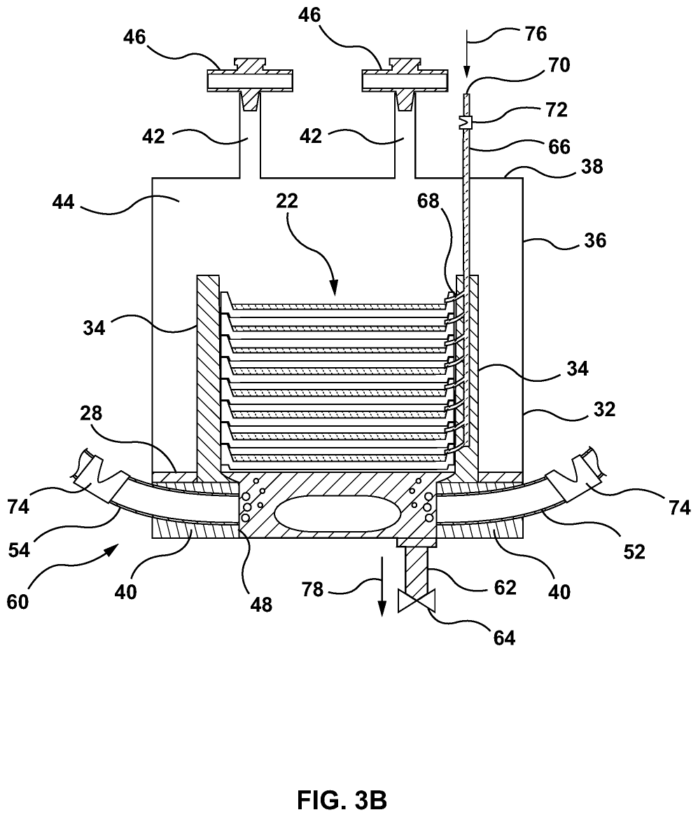

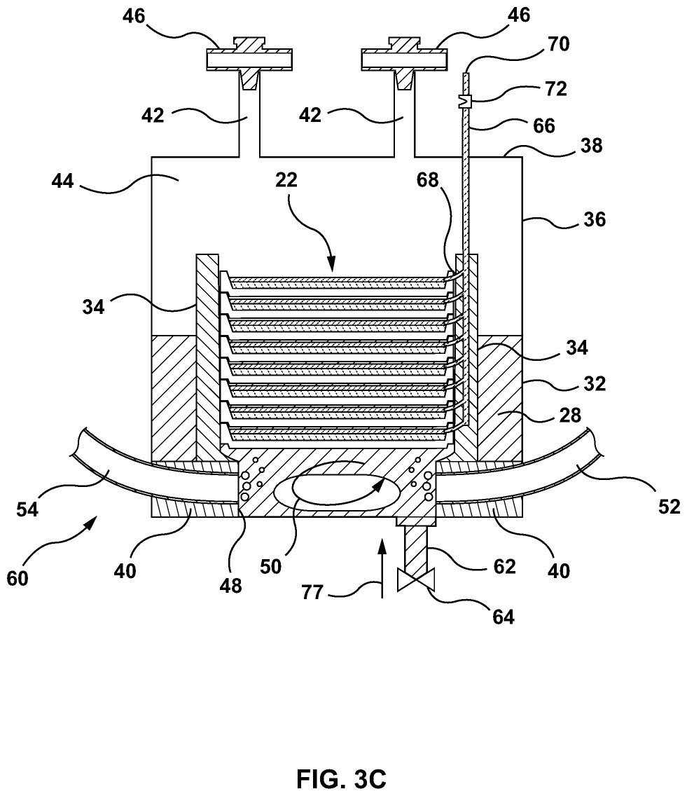

FIGS. 3A, 3B and 3C show a second bioreactor 60. Parts of the second bioreactor 60 having the same reference numeral are similar to parts of the first bioreactor 30. The description of the first bioreactor 30 above applies to the second bioreactor 60 except where described differently below.

The second bioreactor 60 performs the cell seeding in situ. The base 40 of the second bioreactor 60 has a drain 62 with a drain valve 64. The second bioreactor 60 also has a cell seeding manifold 66 with an inlet 70 and a valve 72. The cell seeding manifold 66 also has a set of spigots 68 extending from the manifold 66 past the gaps 20 of the stack 22. Cells, dispersed in a liquid medium, may be conveyed into the inlet 70, through the manifold 66 and through the spigots 68 onto the bottom 16 of the membranes 14 of the stack 22. Optionally, the cell seeding manifold 66 may be used to add media to a tray 10 or to remove (e.g. aspirate) media from a tray 10.

FIG. 3A shows the second bioreactor 60 configured to grow or expand cells in the stack 22. The stack 22 is immersed in a growth medium 28, one or more gasses flow into the base 40, and the impeller 50 is rotated to move or mix gas and liquid through the plenum.

FIG. 3B shows the second bioreactor 60 during cell seeding. To perform the seeding, gas valves 74 or clamps optionally close the gas inlets 52, 54. The impeller 50 is optionally stopped. The drain valve 64 is opened to drain the liquid medium 28 from the plenum at least below the gap 20 of the lowest tray 10. Optionally, the second bioreactor 60 may be tilted to pour liquid medium 28 out of the trays 10 through the gaps 20 or liquid medium 28 may be aspirated from the trays 10. The cell seeding valve 72 is opened and cells in suspended in media are added to the trays 10 through the manifold 66 and the spigots 68. The walls 18 of the membranes 14 contain the cells on the membranes 14 and prevent them from falling to the bottom of the second bioreactor 60 while the cells are adhering to the bottoms 16 of the membranes 14.

FIG. 3C shows the second bioreactor 60 configured for re-filling. After a selected amount of cells have been added, the cell-seeding valve 72 is closed. Once the cells have adhered to the membrane 14, or to a layer of cells previously grown on the membranes 14, liquid medium 28 is moved back into the second bioreactor 60 through the drain 62 and the drain valve 64 is closed. The impeller 50 may be restarted. The gas valves 74 may be opened to restart the flow of one or more gasses into the second bioreactor 60.

The drain 62 and drain valve 64 may also be used to adjust the volume of liquid medium 28 in the second bioreactor 60.

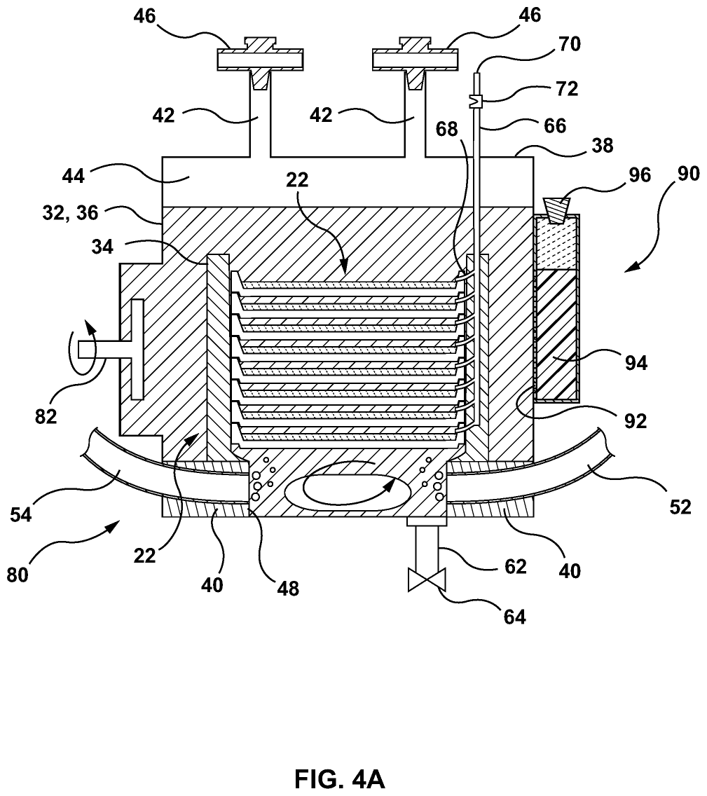

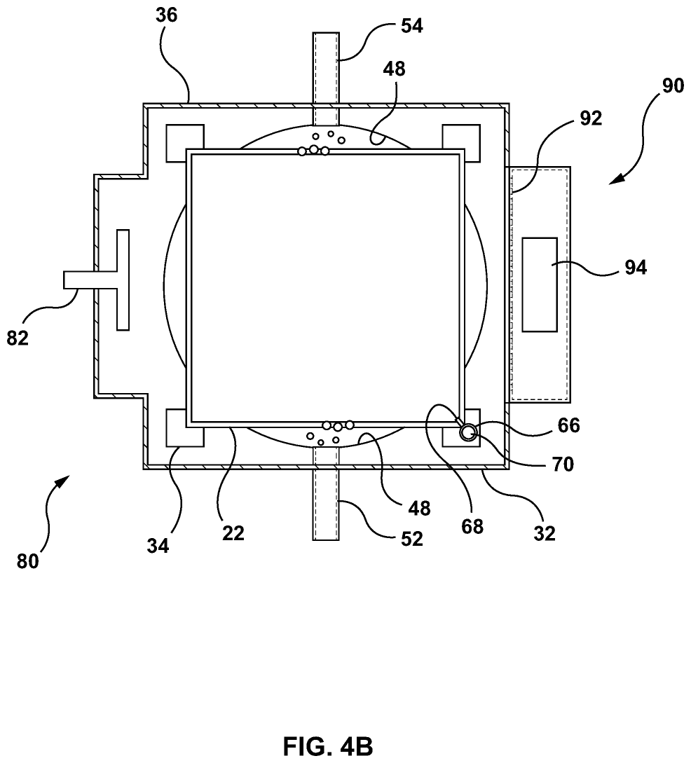

FIG. 4A shows a third bioreactor 80. Parts of the third bioreactor 80 having the same reference numeral are similar to parts of the first bioreactor 30 or the second bioreactor 60. The description of the first bioreactor 30 and second bioreactor 60 above applies to the third bioreactor 80 except where described differently below.

The third bioreactor 80 has a second impeller 82. While the impeller 50 at the bottom of the third bioreactor 80 delivers and distributes gasses, in some examples the impeller 50 might not be sufficient to create a uniform distribution of different agents on the surface of the membranes 14, or to rapidly disperse an added agent into the trays 10. For this purpose, the second impeller 82 is implemented on a side 36 of the third bioreactor 80. In the example shown, the second impeller 82 is in an indentation in a side 36. The second impeller 82 rotates around a horizontal axis. The second impeller 82 may be driven by a shaft passing through the side 36 or by a magnetic coupling. The second impeller 82 pushes the medium through the gaps 20 between the stacked layers 10. Optionally, the impeller 50 and the second impeller 82 may work intermittently, either in phase with each other or at alternating or overlapping intervals.

The third bioreactor 80 also includes a filtration unit or sorption unit 90, optionally as described in U.S. application Ser. No. 17/952,355 Carbon-based Systems for Simultaneous Adsorption and Release of Small Molecules or International Application No, PCT/CA2023/051264 Sorption Unit, Bioreactor with Sorption Unit and Methods of Cell Culture, which are incorporated herein by reference. In the example shown, the filtration or sorption unit is attached to an opening in another wall 36 of the third bioreactor 80. A dialysis membrane 92 separates the filtration or sorption unit 90 from liquid medium 28 in the rest of the third bioreactor 80. The filtration or sorption unit 90 contains a sorption media 94, for example activated carbon. The sorption media 94 can be added to, or removed from, the filtration or sorption unit 90 through a cap 96. The sorption media 94 absorbs lactic acid and ammonia from the liquid media in the third bioreactor 80 and optionally releases small molecules such as glucose or amino acids in the third bioreactor 80. While the impeller 50 is turning, liquid medium 28 may flow tangentially across the dialysis membrane 92. While the second impeller 82 is turning, liquid medium 28 may be pressurized against the dialysis membrane 92. Flow induced by either the impeller 50 or the second impeller 82 may improve the rate of filtration and/or help to wash away any fouling that might have happened on the surface of the dialysis membrane 92 or the sorption media 94.

A bioreactor may be heated through its walls to achieve a temperature of, for example, 32-40 C. Walls of the bioreactor can be heat conductive to transfer heat from a jacket that covers the outside of the bioreactor walls to the inside of the bioreactor.

When media is removed from the bioreactor, it can be heated, cooled, or oxygenated in a chamber connected to the bioreactor, before being returned to the bioreactor.

One method of delivering oxygen to the cells is to periodically remove the media from the bioreactor such that a small amount of the media left on each membrane (i.e. media contained by the wall) is exposed to an oxygen containing gas (e.g. atmospheric gasses). Media may be returned to the bioreactor, e.g. periodically, to avoid drying the media on the membranes, to refresh the media on the membranes, to deliver soluble nutrients to the cells, or to provide a different medium, e.g. to transition between a growth 10 medium and a differentiation medium.

I/We claim:

1. A bioreactor having a plurality of membrane immersed in a plenum, wherein the membranes are adapted for growing adhesion dependent cells, cell sheets, or derivatives of cell sheets.

2. The bioreactor of claim 1 wherein the membranes are provided in trays having wall around the membranes.

3. The bioreactor of claim 1 having a plurality of trays in a stack.

4. The bioreactor of claim 3 having one or more gaps between trays of the stack.

5. The bioreactor of claim 1 having an impeller configured to direct liquid media laterally across the membranes.

6. The bioreactor of claim 1 having a cell seeding system adapted to convey a suspension of cells to a membrane while the membrane is in the bioreactor.

7. The bioreactor of claim 6 wherein cells are provided to the membranes via the gaps.

8. The bioreactor of claim 1 having a sorption system.

9. A method of growing cells sheets comprising steps of seeding a membrane with cells, immersing the membrane in a horizontal orientation in liquid media in a bioreactor, and circulating liquid media in the bioreactor.

10. The method of claim 9 wherein the membrane is seeded with cells in the membrane.

11. The method of claim 9 wherein the circulating media is directed horizontally across the membrane, for example using a secondary homogenizing system.

12. The method of claim 9 comprising adsorbing one or more contaminants from the liquid media.
